(19) United States
(12) Patent Application Publication
(10) Pub. No.: US 2003/0082557 A1
(43) Pub. Date: May 1, 2003

(54) Methods and compositions for mutation detection by liquid chromatography

(76) Inventor: Paul D. Taylor, Gilroy, CA (US)

Correspondence Address: JOHN F. BRADY, TRANSGENOMIC, INC., 2032 CONCOURSE DRIVE, SAN JOSE, CA, 95131, US

(21) Appl. No.: 10/033,104

(22) Filed: Oct. 29, 2001

Related U.S. Application Data
(60) Provisional application No. 60/259,847, filed on Jan. 3, 2001.
(60) Provisional application No. 60/244,436, filed on Oct. 30, 2000.

(51) Int. Cl. C12Q 1/68; C12P 19/34

(52) U.S. Cl. 435/6; 435/91.2

(57) ABSTRACT

The present invention relates to improvements in methods of DNA mutation detection using chromatographic methods such as DHPLC. In particular, the invention relates to methods for increasing the level of heteroduplex during a hybridization process between a DNA fragment and a corresponding wild type DNA fragment by including during the hybridization process nitrogen-containing organic compounds such as betaine, trimethylamine N-oxide, and dimethylglycine. In another aspect the invention provides improved PCR primers for amplifying the DNA to be used in the hybridization. The invention also provides for DNA hybridization kits containing these compounds and primers.

### CROSS REFERENCE TO RELATED CO-PENDING APPLICATIONS

This application is a regular U.S. Patent Application under 35 U.S.C. §111 (a) and claims priority from the following co-pending, commonly assigned provisional applications, each filed under 35 U.S.C. §111 (b), all of which are incorporated herein by reference:

60/259,847 filed Jan. 3, 2001;

60/244,436 filed Oct. 30, 2000.

### FIELD OF THE INVENTION

The present invention concerns improved methods for detection of mutations in nucleic acids. More specifically, the invention concerns methods, compositions, and kits for mutation analysis using denaturing high performance liquid chromatography (DHPLC).

### BACKGROUND OF THE INVENTION

The ability to detect mutations in double stranded polynucleotides, and especially in DNA fragments, is of great importance in medicine, as well as in the physical and social sciences. The Human Genome Project is providing an enormous amount of genetic information which is setting new criteria for evaluating the links between mutations and human disorders (Guyer et al., Proc. Natl. Acad. Sci. U.S.A 92:10841 (1995)). The ultimate source of disease, for example, is described by genetic code that differs from wild type (Cotton, TIG 13:43 (1997)). Understanding the genetic basis of disease can be the starting point for a cure. Similarly, determination of differences in genetic code can provide powerful and perhaps definitive insights into the study of evolution and populations (Cooper, et. al., Human Genetics vol. 69:201 (1985)). Understanding these and other issues related to genetic coding is based on the ability to identify anomalies, i.e., mutations, in a DNA fragment relative to the wild type. A need exists, therefore, for a methodology to detect mutations in an accurate, reproducible and reliable manner.

DNA molecules are polymers comprising sub-units called deoxynucleotides. The four deoxynucleotides found in DNA comprise a common cyclic sugar, deoxyribose, which is covalently bonded to any of the four bases, adenine (a purine), guanine(a purine), cytosine (a pyrimidine), and thymine (a pyrimidine), hereinbelow referred to as A, G, C, and T respectively. A phosphate group links a 3′-hydroxyl of one deoxynucleotide with the 5′-hydroxyl of another deoxynucleotide to form a polymeric chain. In double stranded DNA, two strands are held together in a helical structure by hydrogen bonds between, what are called, complementary bases. The complimentarity of bases is determined by their chemical structures. In double stranded DNA, each A pairs with a T and each G pairs with a C, i.e., a purine pairs with a pyrimidine. Ideally, DNA is replicated in exact copies by DNA polymerases during cell division in the human body or in other living organisms. DNA strands can also be replicated in vitro by means of the Polymerase Chain Reaction (PCR).

Sometimes, exact replication fails and an incorrect base pairing occurs, which after further replication of the new strand results in double stranded DNA offspring containing a heritable difference in the base sequence from that of the parent. Such heritable changes in base pair sequence are called mutations.

In the present invention, double stranded DNA is referred to as a duplex. When the base sequence of one strand is entirely complementary to base sequence of the other strand, the duplex is called a homoduplex. When a duplex contains at least one base pair which is not complementary, the duplex is called a heteroduplex. A heteroduplex can be formed during DNA replication when an error is made by a DNA polymerase enzyme and a non-complementary base is added to a polynucleotide chain being replicated. A heteroduplex can also be formed during repair of a DNA lesion. Further replications of a heteroduplex will, ideally, produce homoduplexes which are heterozygous, i.e., these homoduplexes will have an altered sequence compared to the original parent DNA strand. When the parent DNA has the sequence which predominates in a natural population it is generally called the “wild type.” Many different types of DNA mutations are known. Examples of DNA mutations include, but are not limited to, “point mutation” or “single base pair mutations” wherein an incorrect base pairing occurs. The most common point mutations comprise “transitions” wherein one purine or pyrimidine base is replaced for another and “transversions” wherein a purine is substituted for a pyrimidine (and visa versa). Point mutations also comprise mutations wherein a base is added or deleted from a DNA chain. Such “insertions” or “deletions” are also known as “frameshift mutations”. Although they occur with less frequency than point mutations, larger mutations affecting multiple base pairs can also occur and may be important. A more detailed discussion of mutations can be found in U.S. Pat. No. 5,459,039 to Modrich (1995), and U.S. Pat. No. 5,698,400 to Cotton (1997). These references and the references contained therein are incorporated in their entireties herein.

The sequence of base pairs in DNA codes for the production of proteins. In particular, a DNA sequence in the exon portion of a DNA chain codes for a corresponding amino acid sequence in a protein. Therefore, a mutation in a DNA sequence may result in an alteration in the amino acid sequence of a protein. Such an alteration in the amino acid sequence may be completely benign or may inactivate a protein or alter its function to be life threatening or fatal. Intronic mutations at splice sites may also be causative of disease (e.g. β-thalassemia). Mutation detection in an intron section may be important by causing altered splicing of mRNA transcribed from the DNA, and may be useful, for example, in a forensic investigation.

Detection of mutations is, therefore, of great interest and importance in diagnosing diseases, understanding the origins of disease and the development of potential treatments. Detection of mutations and identification of similarities or differences in DNA samples is also of critical importance in increasing the world food supply by developing diseases resistant and/or higher yielding crop strains, in forensic science, in the study of evolution and populations, and in scientific research in general (Guyer et al., Proc. Natl. Acad. Sci. U.S.A 92:10841 (1995); Cotton, TIG 13:43 (1997)). These references and the references contained therein are incorporated in their entireties herein.

Alterations in a DNA sequence which are benign or have no negative consequences are sometimes called “polymorphisms”. In the present invention, any alterations in the DNA sequence, whether they have negative consequences or not, are called “mutations”. It is to be understood that the method of this invention has the capability to detect mutations regardless of biological effect or lack thereof. For the sake of simplicity, the term “mutation” will be used throughout to mean an alteration in the base sequence of a DNA strand compared to a reference strand. It is to be understood that in the context of this invention, the term “mutation” includes the term “polymorphism” or any other similar or equivalent term of art.

Analysis of DNA samples has historically been done using gel electrophoresis. Capillary electrophoresis has been used to separate and analyze mixtures of DNA. However, these methods cannot distinguish point mutations from homoduplexes having the same base pair length.

Recently, a chromatographic method called ion-pair reverse-phase high pressure liquid chromatography (IP-RP-HPLC), also referred to as Matched Ion Polynucleotide Chromatography (MIPC), was introduced to effectively separate mixtures of double stranded polynucleotides, in general and DNA, in particular, wherein the separations are based on base pair length (Huber, et al., Chromatographia 37:653 (1993); Huber, et al., Anal. Biochem. 212:351 (1993); U.S. Pat. Nos. 5,585,236; 5,772,889; 5,972,222; 5,986,085; 5,997,742; 6,017,457; 6,030,527; 6,056,877; 6,066,258; 6,210,885; and U.S. patent application Ser. No. 09/129,105 filed Aug. 4, 1998.

As the use and understanding of IP-RP-HPLC developed it became apparent that when IP-RP-HPLC analyses were carried out at a partially denaturing temperature, i.e., a temperature sufficient to denature a heteroduplex at the site of base pair mismatch, homoduplexes could be separated from heteroduplexes having the same base pair length (Hayward-Lester, et al., Genome Research 5:494 (1995); Underhill, et al., Proc. Natl. Acad. Sci. U.S.A 93:193 (1996); Doris, et al., DHPLC Workshop, Stanford University, (1997)). These references and the references contained therein are incorporated herein in their entireties. Thus, the use of denaturing high performance liquid chromatography (DHPLC) was applied to mutation detection (Underhill, et al., Genome Research 7:996 (1997); Liu, et al., Nucleic Acid Res., 26;1396 (1998)).

These chromatographic methods are generally used to detect whether or not a mutation exists in a test DNA fragment. In a typical experiment, a test nucleic acid fragment is hybridized with a wild type fragment and analyzed by DHPLC. If the test fragment contains a mutation, then the hybridization product ideally includes both homoduplex and heteroduplex molecules. If no mutation is present, then the hybridization only produces homoduplex wild type molecules. The elution profile of the hybridized test fragment can be compared to a control in which a wild type fragment is hybridized to another wild type fragment. Any change in the elution profile (such as the appearance of new peaks or shoulders) between the hybridized test fragment and the control is assumed to be due to a mutation in the test fragment.

Single nucleotide polymorphisms (SNPs) are thought to be ideally suited as genetic markers for establishing genetic linkage and as indicators of genetic diseases (Landegre et al. Science 242:229-237 (1988)). In some cases a single SNP is responsible for a genetic disease. According to estimates the human genome may contain over 3 million SNPs. Due to their propensity they lend themselves to very high resolution genotyping. The SNP consortium, a joint effort of 10 major pharmaceutical companies, has announced the development of 300,000 SNP markers and their placement in the public domain by mid 2001.

The efficiency of DHPLC for detection of novel mutations (frequently termed scanning) has been quantified by several authors. Results ranged from 87% detection when a single-temperature analysis was used without any amplicon design (Cargill, et al. Nature Genet. 22:231-238 (1999)) to 100% detection in a blinded study of many polymorphisms within a single, well-behaved amplicon (O'Donovan et al., Genomics 52:44-49 (1998)). Comparisons with single-strand conformation polymorphism (SSCP) (Choy et al., Ann. Hum. Genet. 63:383-391 (1999); Gross et al., Hum. Genet. 105:72 78 (1999); Dobson-Stone et al., Eur. J. Hum. Genet. 8:24-32. (2000)) and denaturing gradient gel electrophoresis (DGGE) (Skopek et al., Mutat. Res. 430:13-21 (1999)) have shown DHPLC to have a superior detection rate, whereas most recently DHPLC has been shown to detect mutations reliably in BRCA1 and BRCA2 (Wagner et al., Genomics 62:369-376 (1999)).

A need exists to identify and optimize all the aspects of the DHPLC methodology in order to minimize artifacts and remove ambiguity from the analysis of samples containing putative mutations.

The ability of DHPLC to detect mutations may be less than 100% in some cases. There is a need for methods, compositions, and devices for improving the ability of DHPLC to detect mutations.

### SUMMARY OF THE INVENTION

In one aspect, the present invention concerns a method for preparing a double stranded DNA fragment for mutation detection by denaturing high performance liquid chromatography, the double stranded DNA fragment corresponding to a wild type double stranded DNA fragment having a known nucleotide sequence. In a preferred embodiment, the method includes (a) amplifying a section of the double stranded DNA fragment for mutation detection by PCR, using a set of primers which flank the section, and wherein at least one primer of the set incorporates a sequence comprising solely GC content on its 5′ end; (b) hybridizing the amplification product of step (a) with wild type double stranded DNA corresponding to said section, whereby a mixture comprising one or more heteroduplexes is formed if said section includes a mutation; and (c) including during the hybridizing step an amount of a composition including a nitrogen-containing organic compound as described herein. Examples of the nitrogen-containing compound include compounds according to the formula:

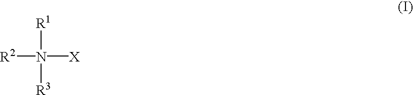

wherein: R¹, R², and R³, may be the same or different and are independently selected from the group consisting of hydrogen, methyl, ethyl, hydroxy ethyl, and propyl, with the proviso that no more than two of R¹, R², and R³ are hydrogen; and

X is a moiety selected from the group consisting of:

radicals of the formulas

═O;

→O

—CH₃;

—CH₂CH₃; and

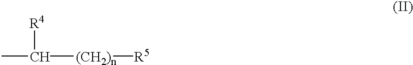

wherein: R⁴ is selected from the group consisting of methyl and hydrogen and, when combined with R¹, forms a pyrrolidine ring; R⁵ is selected from the group consisting of —CO₂H, —CH₂OH, and —SO₃H; and n is an integer of from 0 to 2; with the proviso that, when R¹ and R⁴ form a pyrrolidine ring, no more than one of R² and R³ is hydrogen.

The compound is included during the hybridization in an amount effective to increase the amount of heteroduplex DNA double stranded DNA fragment for mutation detection. Non-limiting examples of such compounds includes trimethylglycine (betaine), bincine, choline, sarcosine, stachydrine, trimethylamine N-oxide, and sulfobetaine. Other compounds include tetraalkylammonium salts such as tetramethylammonium chloride, tetraethylammonium chloride. Other compounds useful in the invention are further described herein.

The compound can be present at a concentration in the range of 1M to 8M. The liquid chromatography is preferably carried out under conditions effective to at least partially denature said heteroduplexes. The double stranded DNA fragment for mutation detection can be unpurified DNA (e.g. a crude cell lysate). In the method at least one of the PCR primers incorporates up to 40 bases of solely GC content on the 5′ end.

Step (b) preferably includes (i) heating the mixture of step (b) to a temperature at which the strands are completely denatured; (ii) cooling the product of step (i) until the strands are completely annealed, whereby a mixture comprising one or more heteroduplexes is formed if said section includes a mutation.

In another aspect, the invention concerns the product of the hybridization method described.

In another aspect, the invention includes a method for mutation detection of a double stranded DNA fragment by denaturing high performance liquid chromatography, the double stranded DNA fragment corresponding to a wild type double stranded DNA fragment having a known nucleotide sequence. In one embodiment, the method includes (a) amplifying a section of the double stranded DNA fragment by PCR using a set of primers which flank the ends of the section, wherein at least one primer of said set incorporates a sequence comprising solely GC content on the 5′ end; (b) hybridizing the amplification product of step (a) with wild type double stranded DNA corresponding to the section, whereby a mixture comprising one or more heteroduplexes is formed if the section includes a mutation; (c) analyzing the product of step (b) by Denaturing High Performance Liquid Chromatography; and (d) including during said hybridizing an amount of a composition comprising a nitrogen-containing compound as described herein and wherein the composition is included in an amount effective to increase the amount of heteroduplex DNA.

In still another aspect, the invention provides a kit or kits for preparing a double stranded DNA for mutation detection by liquid chromatography. This kit can include separate containers containing: one or more PCR primers, wherein at least one primer of said set incorporates a sequence comprising solely GC content on the 5′ end; a nitrogen-containing compound as described herein; a DNA polymerase, preferably a proofreading DNA polymerase; wild type DNA corresponding to the target sequence; a reverse phase separation medium; a liquid chromatography system; instructional material; software for operating the chromatography system; and software for analyzing and modeling the melting properties of the double stranded DNA for mutation detection.

### BRIEF DESCRIPTION OF THE DRAWINGS

FIG. 1 shows a schematic representation of a hybridization to form homoduplex and heteroduplex DNA molecules and the mutation separation profile of the DNA molecules.

FIG. 2. Predicted melting map of homoduplex DYS271 (variant A) unclamped (solid line) and with a 20-base GC-clamp attached (dashed line).

FIG. 3. Predicted melting behavior of the GC-clamped DYS271 fragment.

FIG. 4. Examples of DHPLC profiles showing the effect of GC-clamp and the effect of a nitrogen-containing composition present during the hybridization.

FIG. 5. DHPLC chromatograms showing effect of nitrogen-containing compound present during hybridization process on the yield of heteroduplex.

FIG. 6. DHPLC chromatograms of GC-clamped, hybridized sample of the A and G variants differing at position 168.

FIG. 7. Shows retention times of the peaks in FIG. 6 corresponding to the AC and GT heteroduplex species and the AT and GC homoduplex species. The vertical bars represent peak width at half-height, emphasizing the peak broadening.

FIG. 8. Illustrates a temperature titration showing the progressive denaturation of a mixture of 30T-44G-168A hybridized with 30C-44A-168G in the presence of a nitrogen-containing compound.

### DETAILED DESCRIPTION OF THE INVENTION

A reliable way to detect mutations is by hybridization of the putative mutant strand in a sample with the wild type strand (Lerman, et al., Meth. Enzymol., 155:482 (1987)). If a mutant strand is present, then, typically, two homoduplexes and two heteroduplexes will be formed as a result of the hybridization process. Hence separation of heteroduplexes from homoduplexes provides a direct method of confirming the presence or absence of mutant DNA segments in a sample.

In a general aspect, the instant invention concerns methods and compositions for use during hybridization of DNA molecules for use during a hybridization process prior to mutation analysis.

The instant invention is based in part on the surprising discovery by Applicants that certain nitrogen-containing organic compounds, as disclosed herein, when included during the hybridization process increased the yield of heteroduplex produced during hybridization increased and increased the resolution between homoduplex and heteroduplex molecules, thus facilitating the detection of mutations in DNA by DHPLC. These improvements by the nitrogen-containing compounds were primarily observed when the products of the hybridization included a GC-clamp.

In general aspects, the present invention concerns methods, compositions, kits and devices for preparing a sample for analysis by DHPLC. In particular, Applicants have discovered that certain compounds and compositions, as will be described herein, when included during the hybridization step, markedly increase the detectability of mutations as analyzed using DHPLC.

The mutation analysis involves a DNA separation process and can be performed by a variety of methods, such as liquid chromatography (LC), capillary electrophoresis (CE), and denaturing gradient gel electrophoresis (DGGE).

Examples of suitable liquid chromatographic methods include IP-RP-HPLC and ion exchange chromatography where these are performed under partially denaturing conditions. The use of ion exchange chromatography is disclosed in U.S. patent application Ser. No. 09/756,070 filed Jan. 6, 2001. For purposes of clarity and not by way of limitation, DHPLC is described herein.

The term “nucleic acids”, as used herein, refers to either DNA or RNA. It includes plasmids, infectious polymers of DNA and/or RNA, nonfunctional DNA or RNA, chromosomal DNA or RNA and DNA or RNA synthesized in vitro (such as by the polymerase chain reaction). “Nucleic acid sequence” or “polynucleotide sequence” refers to a single- or double-stranded polymer of deoxyribonucleotide or ribonucleotide bases read from the 5′ to the 3′ end.

The term “DNA molecule” as used herein refers to DNA molecules in any form, including naturally occurring, recombinant, or synthetic DNA molecules. The term includes plasmids, bacterial and viral DNA as well as chromosomal DNA. The term encompasses DNA fragments produced by cell lysis or subsequent manipulation of DNA molecules. Unless specified otherwise, the left hand end of single-stranded DNA sequences is the 5′ end.

The term “complementary” as used herein includes reference to a relationship between two nucleic acid sequences. One nucleic acid sequence is complementary to a second nucleic acid sequence if it is capable of forming a duplex with the second nucleic acid, wherein each residue of the duplex forms a guanosine-cytidine (G-C) or adenosine-thymidine (A-T) basepair or an equivalent basepair. Equivalent basepairs can include nucleoside or nucleotide analogues other than guanosine, cytidine, adenosine, or thymidine, which are capable of being incorporated into a nucleic acid by a DNA or RNA polymerase on a DNA template. A complementary DNA sequence can be predicted from a known sequence by the normal basepairing rules of the DNA double helix (see Watson J. D., et al. (1987) Molecular Biology of the Gene, Fourth Edition, Benjamin Cummings Publishing Company, Menlo Park, Calif., pp. 65-93). Complementary nucleic acids may be of different sizes. For example, a smaller nucleic acid may be complementary to a portion of a larger nucleic acid.

The terms “purified DNA” or “purified DNA molecule,” as used herein, include reference to DNA that is not contaminated by other biological macromolecules, such as RNA or proteins, or by cellular metabolites. Purified DNA contains less than 5% contamination (by weight) from protein, other cellular nucleic acids and cellular metabolites. The terms “unpurified DNA” or “unpurified DNA molecules” refer to preparations of DNA that have greater than 5% contamination from other cellular nucleic acids, cellular proteins and cellular metabolites. Unpurified DNA may be obtained by using a single purification step, such as precipitation with ethanol combined with either LiCl or polyethylene glycol. The term “crude cell lysate preparation” or “crude cell lysate” or “crude lysate” refers to an unpurified DNA preparation where cells or viral particles have been lysed but where there has been no further purification of the DNA.

Depending on the conditions, ion-pair reverse-phase high performance liquid chromatography (IP-RP-HPLC) separates double stranded polynucleotides by size or by base pair sequence and is therefore a preferred separation technology for detecting the presence of particular fragments of DNA of interest. IP-RP-HPLC is also referred to in the art as “Matched Ion Polynucleotide Chromatography” (MIPC).

The term “chromatographic elution profile” as used herein is defined to include the data generated by the IP-RP-HPLC method when this method is used to separate double stranded DNA fragments. The chromatographic profile can be in the form of a visual display, a printed representation of the data or the original data stream.

IP-RP-HPLC as used herein includes a chromatographic process for separating single and double stranded polynucleotides using non-polar separation media, wherein the process uses a counterion agent, and an organic solvent to release the polynucleotides from the separation media. IP-RP-HPLC separations can be completed in less than 10 minutes, and frequently in less than 5 minutes. IP-RP-HPLC systems (e.g., the WAVE® DNA Fragment Analysis System, Transgenomic, Inc. San Jose, Calif.) are preferably equipped with computer controlled ovens which enclose the columns. Mutation detection at the temperature required for partial denaturation (melting) of the DNA at the site of mutation can therefore be easily performed. The system used for IP-RP-HPLC separations is rugged and provides reproducible results. It is preferably computer controlled and the entire analysis of multiple samples can be automated. The system preferably offers automated sample injection, data collection, choice of predetermined eluting solvent composition based on the size of the fragments to be separated, and column temperature selection based on the base pair sequence of the fragments being analyzed. The separated mixture components can be displayed either in a gel format as a linear array of bands or as an array of peaks. The display can be stored in a computer storage device. The display can be expanded and the detection threshold can be adjusted to optimize the product profile display. The reaction profile can be displayed in real time or retrieved from the storage device for display at a later time. A mutation separation profile, a genotyping profile, or any other chromatographic separation profile display can be viewed on a video display screen or as hard copy printed by a printer.

The term “temperature titration” of DNA as used herein includes an experimental procedure in which the retention-time from DHPLC is plotted as the ordinate against column temperature as the abscissa.

A “homoduplex” is defined herein to include a double stranded DNA fragment wherein the bases in each strand are complementary relative to their counterpart bases in the other strand.

A “heteroduplex” is defined herein to include a double stranded DNA fragment wherein at least one base in each strand is not complementary to at least one counterpart base in the other strand. Since at least one base pair in a heteroduplex is not complementary, it takes less energy to separate the bases at that site compared to its fully complementary base pair analog in a homoduplex. This results in the lower melting temperature at the site of a mismatched base of a heteroduplex compared to a homoduplex. A heteroduplex can be formed by annealing of two nearly complementary sequences.

The term “hybridization” refers to a process of heating and cooling a dsDNA sample, e.g., heating to 95° C. followed by slow cooling. The heating process causes the DNA strands to denature. Upon cooling, the strands re-combine, or anneal, into duplexes.

The term “heteromutant” is defined herein to include a DNA fragment containing a polymorphism or non-complementary base pair.

In the operation of the DHPLC method, the determination of a mutation is preferably made by hybridizing the homozygous sample with the known wild type fragment and performing a DHPLC analysis at a partially denaturing temperature. If the sample contained only wild type fragments then a single peak would be seen in the DHPLC analysis since no heteroduplexes could be formed. In the operation of the DHPLC method, the determination of a mutation can be made by hybridizing the homozygous sample with the corresponding wild type fragment and performing a DHPLC analysis. If the sample contained only wild type fragments then a single peak would be seen in the DHPLC analysis since no heteroduplexes could be formed. If the sample contained homozygous mutant fragments or was heterozygous for the mutation, then analysis by DHPLC can be used to detect the separation of homoduplexes and heteroduplexes.

When mixtures of DNA fragments are mixed with an ion pairing agent and applied to a reverse phase separation column, they are separated by size, the smaller fragments eluting from the column first. However, when IP-RP-HPLC is performed at an elevated temperature which is sufficient to denature that portion of a DNA fragment domain which contains a heteromutant site, then heteroduplexes separate from homoduplexes. IP-RP-HPLC, when performed at a temperature which is sufficient to partially denature a heteroduplex, is referred to as DHPLC. DHPLC is also referred to in the art as “Denaturing Matched Ion Polynucleotide Chromatography” (DMIPC).

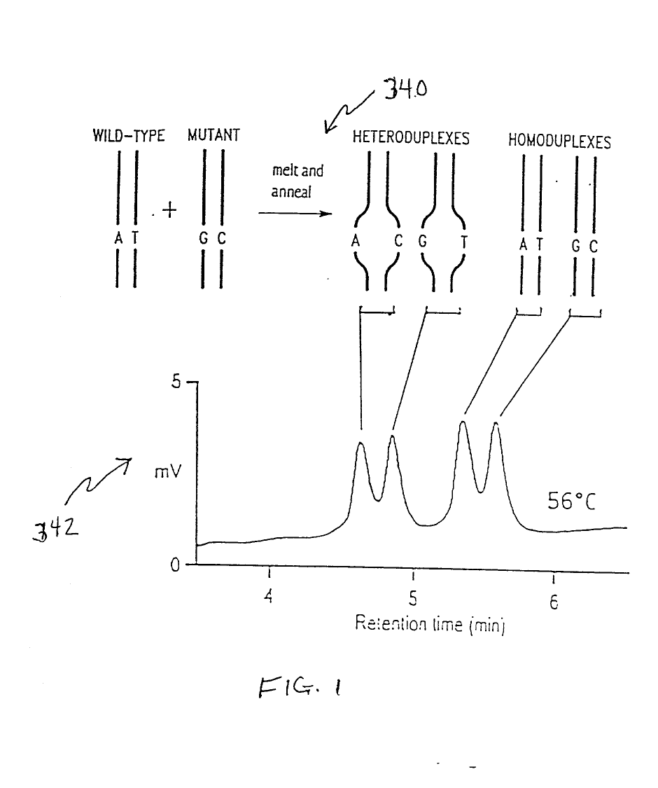

The term “mutation separation profile” is defined herein to include a DHPLC separation chromatogram which shows the separation of heteroduplexes from homoduplexes. Such separation profiles are characteristic of samples which contain mutations or polymorphisms and have been hybridized prior to being separated by DHPLC. The DHPLC separation chromatogram shown in FIG. 1 exemplifies a mutation separation profile as defined herein.

FIG. 1 illustrates the temperature dependent separation of a mixture of homoduplexes and heteroduplexes by DHPLC. The data in FIG. 1 were obtained from a mixture containing both 209 bp homoduplex mutant and 209 bp homoduplex wild type species. Such “mutation standards” provide a mixture of DNA species that when hybridized and analyzed by DHPLC, produce previously characterized mutation separation profiles which can be used to evaluate the performance of the chromatography system. Mutation standards can be obtained commercially (e.g. a WAVE Optimized™ UV 209 bp Mutation Standard (part no. 700210), GCH338 Mutation Standard (part no. 700215), and HTMS219 Mutation Standard (part no. 700220) are available from Transgenomic, Inc. and a 209 bp mutation standard is also available from Varian, Inc.). Prior to injection of the mixture onto the separation column, the mutation standard was hybridized as shown in the scheme 340. The hybridization process created two homoduplexes and two heteroduplexes. As shown in the mutation separation profile 342, the hybridization product was separated using DHPLC. The two lower retention time peaks represent the two heteroduplexes and the two higher retention time peaks represent the two homoduplexes. The two homoduplexes separate because the A-T base pair denatures at a lower temperature than the C-G base pair. Without wishing to be bound by theory, the results are consistent with a greater degree of denaturation in one duplex and/or a difference in the polarity of one partially denatured heteroduplex compared to the other, resulting in a difference in retention time on the reverse-phase separation column.

However, in some cases, only two peaks or partially resolved peaks are observed in DHPLC analysis. The two homoduplex peaks may appear as one peak or a partially resolved peak and the two heteroduplex peaks may appear as one peak or a partially resolved peak. In some cases, only a broadening of the initial peak is observed.

In preparing a set of DNA fragments for analysis by DHPLC, it is usually assumed that all of the fragments have the same length since they are generated using the same set of PCR primers. It is further usually assumed that the fragments are eluted under essentially the same conditions of temperature and solvent gradient. The pattern or shape of the mutation separation profile consists of peaks representing the detector response as various species elution during the separation process. The profile is determined by, for example, the number, height, width, symmetry and retention time of peaks. Other patterns can be observed, such as 3 or 2 peaks. The profile can also include poorly resolved shoulders. The shape of the profile contains useful information about the nature of the sample. The pattern or shape of the resulting chromatogram will be influenced by the type and location of the mutation. Each mutation (e.g. single nucleotide polymorphism (SNP)) has a corresponding elution profile, or signature, at a given set of elution conditions of temperature and gradient.

An advantage of the instant invention, as will be shown hereinbelow, is that it can improve the resolution between heteroduplex and homoduplex peaks even for mutations that are difficult to detect.

Detection of unknown mutations requires a highly sensitive, reproducible and accurate analytical method. The design of polymerase chain reaction (PCR) primers used to amplify DNA samples which are to be analyzed for the presence of mutations is an important factor contributing to accuracy, sensitivity and reliability of mutation detection. The design of primers specifically for the purpose of enhancing and optimizing mutation detection by DHPLC is disclosed in U.S. Pat. No. 6,287,822, the PCT publication WO9907899, by Xiao et al. (Human Mutation 17:439-474 (2001) and by Kuklin et al., (Genet. Test. 1:201-206 (1998).

Generally, a fragment, such as an exon, will contain sample sequences, or sections, having different melting temperatures, but which have a narrow range of variation within any one section.

The change in the structure of DNA from an orderly helix to a disordered, unstacked structure without base pairs is called the helix-random chain transition, or melting. Statistical-mechanical analysis of equilibria representing this change as a function of temperature for double-stranded molecules of natural sequence has been presented by Wartell and Montroll ((Adv. Chem. Phys. 22: 129 (1972)) and by Poland (1974). The theory assumes that each base pair can exist in only two possible states-either stacked, helical, and hydrogen bonded, or disordered. It permits calculation of the probability that each individual base pair is either helical or melted at any temperature, given only the base sequence and a very small number of empirically calibrated parameters. The statistical-mechanical theories take into account the differing intrinsic stabilities of each base pair or cluster of neighboring base pairs, the influence of adjacent helical structure on the probability that a neighboring base pair is helical or melted (the cooperativity), and the restrictions on the conformational liberty of a disordered region if it is bounded at both ends by helical regions. Poland (Cooperative Equilibria in Physical Biochemistry, Oxford Univ. Press, Oxford, England, (1978)) has presented a relatively accessible explanation of the theory and its development from simple principles. Wartell and Benight (Phys. Rep. 126: 67 (1985)) have recently reviewed the theory and presented a careful comparison of theoretical and experimental results. A more general survey has been presented by Gotoh (Adv. Biophys. 16:1 (1983)). Since the theory is based on distribution of each base pair between only two states, it does not take into account patterns of pairing between the two strands that do not occur in the original helix, nor pairing within sections of the separated strands. The relevance of such considerations has not yet been demonstrated, but they can be imagined to occur as melting intermediates in relatively long molecules where the calculated and experimental results may show significant discrepancies. Apparent departure of experimental results from theoretical expectation occurs for some sequences because of exceedingly slow approach to equilibrium (Suyama et al. Biopolymers 23:409 (1984); Anshelevich et al, Biopolymers 23:39 (1984)).

Iteration of the probability calculation at a closely spaced series of temperature steps and interpolation permit determination of the midpoint temperature at which each base pair is at 50/50 equilibrium between the helical and melted states. The MELT program provides the midpoint temperature and some other functions. A plot of midpoint temperature as a function of position along the molecule is called a melting map. It clearly shows that the melting of nearby base pairs is closely coupled over substantial lengths of the molecule despite their individual differences in stability. The existence of fairly long regions, 30-300 bp, termed domains, in which all bases melt at very nearly the same temperature, is typical. The melting map directly delineates the lowest melting domains in the molecules.

In the instant invention, a selected section of a target DNA fragment is amplified by PCR using both forward and reverse primers which flank the first and second ends of the section. Applicants have found that mutation detection of dsDNA using DHPLC is more reliable and accurate if the mutation is located within a section having a narrow midpoint temperature range. An example of such a section is the constant melting domain as described by Lerman et al. (Meth. Enzymol. 155:482 (1987)).

When the sequence of a DNA fragment to be amplified by PCR is known, commercially available software can be used to design primers which will produce either the whole fragment, or any section, within the fragment. The melting map of a fragment can be constructed using software such as MacMelt (BioRad Laboratories, Hercules, Calif.), MELT (Lerman et al. Meth. Enzymol. 155:482 (1987)), or WinMelt (BioRad Laboratories).

In the instant invention, the “melting point-50”, or midpoint temperature, of a base pair is defined to include that temperature at which the base pair is 50% helical, i.e., in 50/50 equilibrium between the helical and melted states. For a DNA sequence, the melting point-50 can be plotted as a function of the base position. This plot is called a melting map and can be generated, for example, using the MELT program as described hereinabove.

In another embodiment, the “melting point-75” can be plotted in the melting map. The melting point-75 is the temperature at which a base is 75% helical, i.e. in 75/25 equilibrium between the helical and melted states.

In general, a “melting point-N” can be used where N represents the temperature at which a base is N% helical, i.e., in N/(100-N) equilibrium between the helical and melted states. In this aspect of the invention, N can range from about 10 to about 90, and preferably about 20 to about 80. An optimal value for N can be determined empirically. Examples of preferred values for N are 75 and 50, which can be used in the MELT program, and which have been found to be useful in preparing PCR primers as described herein.

The primers for use in the instant invention are preferably selected to amplify a section of the target DNA fragment in which the bases have a narrow range of melting point-75. For example, the range can be less that about 15° C.

DHPLC, as known in the art, provides a method for separating heteroduplex and homoduplex nucleic acid molecules (e.g., DNA or RNA) in a mixture using high performance liquid chromatography. In the separation method, a mixture containing both heteroduplex and homoduplex nucleic acid molecules is applied to a stationary reverse-phase support. The sample mixture is then eluted with a mobile phase containing an ion-pairing reagent and an organic solvent. Sample elution is carried out under conditions effective to at least partially denature the heteroduplexes and results in the separation of the heteroduplex and homoduplex molecules.

Stationary phases for carrying out the separation include reverse-phase supports composed of alkylated base materials, such as silica, polyacrylamide, alumina, zirconia, polystyrene, and styrene-divinyl copolymers. Styrene-divinyl copolymer base materials include copolymers composed of i) a monomer of styrene such as styrene, alkyl-substituted styrenes, α-methylstyrene, or alkyl substituted α-methylstyrenes and ii) a divinyl monomer such as divinylbenzene or divinylbutadiene. In one embodiment, the surface of the base material is alkylated with hydrocarbon chains containing from about 4-18 carbon atoms. In another embodiment, the stationary support is composed of beads from about 1-100 microns in size.

Examples of suitable separation media are described in the following U.S. patents and patent applications: U.S. Pat. Nos. 6,056,877; 6,066,258; 5,453,185; 5,334,310; U.S. patent application Ser No. 09/493,734 filed Jan. 28, 2000; U.S. patent application Ser No. 09/562,069 filed May 1, 2000; and in the following PCT applications: WO98/48914; WO98/48913; PCT/US98/08388; PCT/US00/11795.

An example of a suitable column based on a polymeric stationary support is the DNASep® column (available from Transgenomic). An example of a suitable column based on a silica stationary support is the Microsorb Analytical column (Varian and Rainin).

Monolithic columns, including capillary columns, can also be used, such as disclosed in U.S. Pat. No. 6,238,565; U.S. patent application Ser. No. 09/562,069 filed May 1, 2000; the PCT application WO0015778; and by Huber et al (Anal. Chem. 71:3730-3739 (1999)).

The length and diameter of the separation column, as well as the system mobile phase pressure and temperature, and other parameters, can be varied as is known in the art.

Size-based separation of DNA fragments can also be performed using batch methods and devices as disclosed in U.S. Pat Nos. 6,265,168; 5,972,222; and 5,986,085.

In DHPLC, the mobile phase contains an ion-pairing agent (i.e. a counterion agent) and an organic solvent. Ion-pairing agents for use in the method include lower primary, secondary and tertiary amines, lower trialkylammonium salts such as triethylammonium acetate and lower quaternary ammonium salts. Typically, the ion-pairing reagent is present at a concentration between about 0.05 and 1.0 molar. Organic solvents for use in the method include solvents such as methanol, ethanol, 2-propanol, acetonitrile, and ethyl acetate.

In one embodiment, the mobile phase for carrying out the separation of the present invention contains less than about 40% by volume of an organic solvent and greater than about 60% by volume of an aqueous solution of the ion-pairing agent. In a preferred embodiment, elution is carried out using a binary gradient system.

At least partial denaturation of heteroduplex molecules can be carried out several ways including the following. Temperatures for carrying out the separation method of the invention are typically between about 40° and 70° C., preferably between about 55°-65° C. In a preferred embodiment, the separation is carried out at 56° C. Alternatively, in carrying out a separation of GC-rich heteroduplex and homoduplex molecules, a higher temperature (e.g., 64° C.) is preferred.

A wide variety of liquid chromatography systems are available that can be used for conducting DHPLC. These systems typically include software for operating the chromatography components, such as pumps, heaters, mixers, fraction collection devices, injector. Examples of software for operating a chromatography apparatus include HSM Control System (Hitachi), ChemStation (Agilent), VP data system (Shimadzu), Millennium32 Software (Waters), Duo-Flow software (Bio-Rad), and ProStar Biochromatography HPLC System (Varian).

Examples of preferred liquid chromatography systems for carrying out DHPLC include the WAVE DNA Fragment Analysis System (Transgenomic) and the Varian ProStar Helix™ System (Varian).

In carrying out DHPLC analysis, the operating temperature and the mobile phase composition can be determined by trial and error. However, these parameters are preferably obtained by using software. Computer software that can be used in carrying out DHPLC is disclosed in the following patents and patent applications: U.S. Pat. No. 6,287,822; 6,197,516; U.S. patent application Ser. No. 09/469,551 filed Dec. 22,1999; and in WO0146687 and WO0015778. Examples of software for predicting the optimal temperature for DHPLC analysis are disclosed by Jones et al. in Clinical Chem. 45:113-1140 (1999) and in the website having the address of http://insertion.stanford.edu/melt.html. And example of a commercially available software includes WAVEMaker™ software (Transgenomic, Inc.).

An important aspect of the present invention concerns compounds that improve the detection of mutations in DNA. The compounds used in the present invention are nitrogen-containing organic molecules that are capable of increasing the level of heteroduplex formation in a hybridization process as described herein. Preferred embodiments of these compounds are represented by the formula:

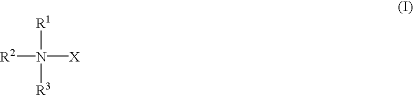

wherein:

R¹, R², and R³, may be the same or different and are independently selected from the group consisting of hydrogen, methyl, ethyl, hydroxyethyl, and propyl, with the proviso that no more than two of R¹, R², and R³ are hydrogen; and

X is a moiety selected from the group consisting of: radicals of the formulas

═O;

→O

—CH₃;

—CH₂CH₃; and

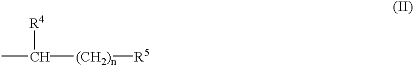

wherein:

R⁴ is selected from the group consisting of methyl and hydrogen and, when combined with R¹, forms a pyrrolidine ring;

R⁵ is selected from the group consisting of —CO₂H, —CH₂OH, and—SO₃H; and

n is an integer of from 0 to 2; and

with the proviso that, when R¹ and R⁴ form a pyrrolidine ring, no more than one of R² and R³ is hydrogen; and

wherein the composition is included in an amount effective to increase the amount of heteroduplex DNA.

When a pyrrolidine ring is formed by R¹ and R⁴′ a compound of formula III is formed.

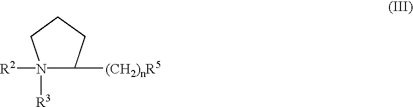

In certain preferred embodiments, the methods and kits of this invention use compounds of formula I wherein R¹, R², and R³, may be the same or different and are independently selected from the group consisting of hydrogen, methyl, ethyl, and propyl, with the proviso that no more than two of R¹, R², and R³ are hydrogen and, when R¹ and R⁴ form a pyrrolidine ring, no more than one of R² and R³ is hydrogen.

In another group of preferred embodiments, the methods and kits of this invention use a compound of formula I wherein X is —CH₂CO₂H. Further preferred embodiments within this group use compounds where R¹, R² and R³ are methyl; where R¹, R² are methyl and R³ is hydrogen; or where R¹ is methyl and R² and R³ are hydrogen.

In further preferred embodiments, the methods and kits of this invention use a compound of formula I wherein X is ═0 and R¹, R² and R³ are methyl.

In still further preferred embodiments, the methods and kits of this invention use a compound of formula I wherein R¹ and R⁴ form a pyrrolidine ring, R² and R³ are methyl, n is 0, and R⁵ is —CO₂H (stachydrine, formula IV).

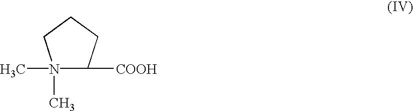

In yet another group of preferred embodiments, the methods and kits of this invention use compounds R¹, R² and R³ are methyl and X is —CH₂—SO₃H (sulfobetaine).

In general, the compounds as described herein are commercially available. For example, betaine, choline, dimethylglycine, sarcosine, and trimethylamine N-oxide can all be obtained from Sigma-Aldrich Corp.

These compounds may also be synthesized by routine methods known to those of skill in the art. For example, compounds of formula wherein R⁴ is H, n is 0 and R⁵ is —CO₂H can be synthesized by the method of Lloyd, et al. (1992) J. Pharm. Pharmacol. 44:507-511. In general, ethyl chloroacetate is heated to reflux with the appropriate tertiary amine in ethanol. When the reaction is complete, the ethanol is removed from the reaction mixture by evaporation under reduced pressure. The residue is dissolved in 3-6% w/v aqueous HCI and warmed to reflux. Evaporation of the solvent under reduced pressure provides the desired products. Typically, these products can be recrystallized from an acetonitrile/water mixture.

Compounds of formula I wherein R⁴ is H or CH₃, n is 1 and R⁵ is CO₂H can be synthesized by the method of Fiedorek, F. T., U.S. Pat. No. 2,548,428. In brief, betalactones are reacted with tertiary amines to provide the desired compounds.

Compounds of formula I wherein R⁴ is H, n is 2, and R⁵ is —CO₂H can be synthesized by the method of Aksnes, G., et al. J. Chem. Soc. London 1959:103-107. In brief, 4-bromobutyric acid (Sigma-Aldrich) is converted to a methyl ester by treatment with methyl alcohol and catalytic sulfuric acid. Subsequent treatment of the methyl ester with excess alcoholic tertiary amine provides the desired compounds.

Compounds of formula I wherein R⁴ and R¹ are taken together to form a pyrrolidine ring and where R⁵ is CO₂H are synthesized by the general method of Karer, et al. (1925) Helv. Chim. Acta. 8:364. For example, stachydrine is formed by the methylation of proline, according to this procedure.

Compounds of formula I wherein X is ═O are synthesized by oxidation of the corresponding tertiary amines (see March, J. (1992) Advanced Organic Chemistry, Reactions, Mechanisms and Structure, Fourth Edition, John Wiley and Sons, New York, pp.1200-1201). Typically, the oxidation is carried out with hydrogen peroxide, but other peracids may also be used.

Compounds of formula I wherein X is →O include N-oxides. The “→” symbol indicates a dative bond.

Sulfobetaine can be synthesized according to the procedure of King, J. F., et al. (1985) J. Phosphorus Sulfur 25:11-20. Other compounds of formula I wherein R⁵ is —SO₃H can also be synthesized by modifications of this procedure and by other methods known to those of skill in the art.

Examples of these compounds include betaine, bicine, choline, trimethylamine N-oxide, dimethylglycine, tetrapropylammoinium chloride (TPACl), tetraethylammonium chloride (TEACl), tetramethylammonium chloride (TMACl).

Some nitrogen-containing compounds of the present invention may be present with a positive or negative charge or with both a positive and negative charges, depending on the pH of the solution. It is understood that these various forms of these compounds are included in the present invention.

The term betaine, as used herein, refers to N,N,N-trimethylglycine.

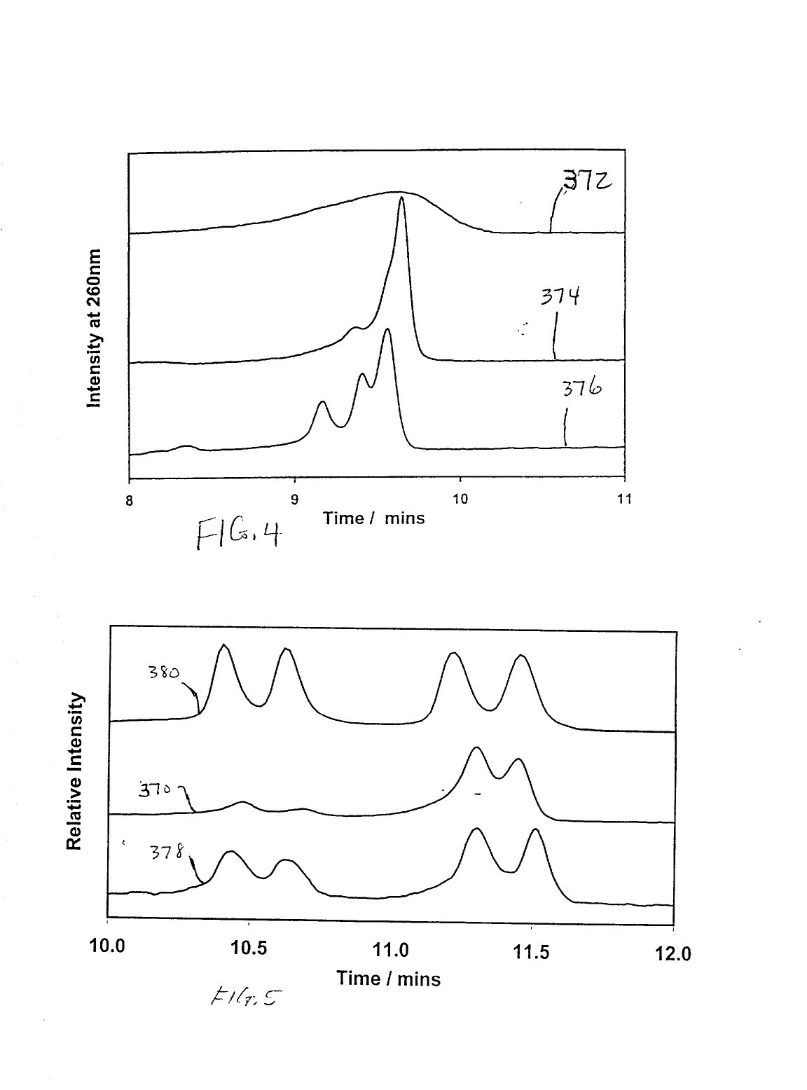

Compounds useful in increasing the level of heteroduplex in a hybridization process are described herein. These compounds may be tested for their relative ability to increase heteroduplex formation. In a specific example, the compounds may be tested by the procedure described in the Examples hereinbelow in relation to FIGS. 4 and 5. More generally, in an embodiment of this aspect of the invention, a preparation of a double stranded DNA fragment is mixed with corresponding wild type DNA in the presence or absence of a selected concentration of a nitrogen-containing compound of the invention and subjected to hybridization. The double stranded DNA fragment and the corresponding wild type DNA have preferably been prepared (e.g. by PCR primer design) such that they each include a GC-clamp and the GC-clamp is therefore also included into the hybridization product. The hybridization product is analyzed by DHPLC. Applicants have found that they are able to detect heteroduplex molecules when they constitute at least about 20% of total DNA molecules after the hybridization process. Therefore, an “effective concentration” for each of the nitrogen-containing compounds is that concentration which yields heteroduplex molecules that constitute at least 10% of the total DNA molecules after the hybridization process when the preparation of a double stranded DNA fragment contains a mutation. Effective concentrations for each of the compounds may be determined by this procedure.

Optimal concentrations for a given compound may vary for different DNA fragments or sites of mutation. These concentrations may be readily determined experimentally by adding different amounts of a compound and determining the level of heteroduplex formed.

In some embodiments, the concentration of the compound during the hybridization can be a selected value within the range from about 0.1 M to about 10M. Examples of preferred concentrations are in the range of about 1 to about 8M, and most preferably in the range of about 2M to about 5M.

An exemplary DNA fragment was been used in illustrating aspects of the instant invention as described in the Examples herein. This fragment comprises a 209-bp fragment from the human Y chromosome locus DYS217 (GenBank accession number S76940). This fragment was selected merely for the purpose of illustrating a difficult to detect mutation. The instant invention is applicable to any fragment that can be analyzed using DHPLC.

In an exemplary embodiment of the method of the instant invention, betaine is included during the hybridization process. When 3M betaine was present during hybridization procedure involving a difficult to detect mutation (as described in the Examples herein), Applicants surprisingly observed a fourfold increased yield of heteroduplex.

Another aspect of the invention concerns the design of PCR primers. In embodiments of the invention any one of the nitrogen-containing compounds as described herein, or a mixture thereof, can be added just prior to the hybridization process, or can be present both during a preceding PCR process and also during the hybridization process.

The present invention involves nucleic acid amplification procedures, such as PCR, which involve chain elongation by a DNA polymerase. There are a variety of different PCR techniques which utilize DNA polymerase enzymes, such as Taq polymerase. See PCR Protocols: A Guide to Methods and Applications. (Innis, M, Gelfand, D., Sninsky, J. and White, T., eds.), Academic Press, San Diego (1990) for detailed description of PCR methodology. In a typical PCR protocol, a target nucleic acid, two oligonucleotide primers (one of which anneals to each strand), nucleotides, polymerase and appropriate salts are mixed and the temperature is cycled to allow the primers to anneal to the template, the DNA polymerase to elongate the primer, and the template strand to separate from the newly synthesized strand. Subsequent rounds of temperature cycling allow exponential amplification of the region between the primers.

There are a variety of different DNA polymerase enzymes that can be used in the invention, although proof-reading polymerases are preferred. DNA polymerases useful in the present invention may be any polymerase capable of replicating a DNA molecule. Preferred DNA polymerases are thermostable polymerases, which are especially useful in PCR. Thermostable polymerases are isolated from a wide variety of thermophilic bacteria, such as Thermus aquaticus (Taq), Thermus brockianus (Tbr), Thermus flavus (Tfl), Thermus ruber (Tru), Thermus thermophilus (Tth), Thermococcus litoralis (Tli) and other species of the Thermococcus genus, Thermoplasma acidophilum (Tac), Thermotoga neapolitana (Tne), Thermotoga maritima (Tma), and other species of the Thermotoga genus, Pyrococcus furiosus (Pfu), Pyrococcus woesei (Pwo) and other species of the Pyrococcus genus, Bacillus sterothermophilus (Bst), Sulfolobus acidocaldarius (Sac) Sulfolobus solfataricus (Sso), Pyrodictium occultum (Poc), Pyrodictium abyssi (Pab), and Methanobacterium thermoautotrophicum (Mth), and mutants, variants or derivatives thereof.

Several DNA polymerases are known in the art and are commercially available (e.g., from Boehringer Mannheim Corp., Indianapolis, Ind.; Life Technologies, Inc.,Rockville, Md.; New England Biolabs, Inc., Beverley, Mass.; Perkin Elmer Corp., Norwalk, Conn.; Pharmacia LKB Biotechnology, Inc., Piscataway, N.J.; Qiagen, Inc., Valencia, Calif.; Stratagene, La Jolla, Calif.). Preferably the thermostable DNA polymerase is selected from the group of Taq, Tbr, Tfl, Tru, Tth, Tli, Tac, Tne, Tma, Tih, Tfi, Pfu, Pwo, Kod, Bst, Sac, Sso, Poc, Pab, Mth, Pho, ES4, VENT™, DEEPVENT™, PFUTurbo™, AmpliTaq®, and active mutants, variants and derivatives thereof. It is to be understood that a variety of DNA polymerases may be used in the present invention, including DNA polymerases not specifically disclosed above, without departing from the scope or preferred embodiments thereof.

Oligonucleotide primers useful in the present invention may be any oligonucleotide of two or more nucleotides in length. Preferably, PCR primers are about 15 to about 30 bases in length, and are not palindromic (self-complementary) or complementary to other primers that may be used in the reaction mixture. Oligonucleotide primers are oligonucleotides used to hybridize to a region of a target nucleic acid to facilitate the polymerization of a complementary nucleic acid. Any primer may be synthesized by a practitioner of ordinary skill in the art or may be purchased from any of a number of commercial venders (e.g., from Boehringer Mannheim Corp., Indianapolis, Ind.; New England Biolabs, Inc., Beverley, Mass.; Pharmacia LKB Biotechnology, Inc., Piscataway, N.J.). It will be recognized that the PCR primers can include covalently attached groups, such as fluroescent tags. U.S. Pat. No. 6,210,885 describes the use of such tags in mutation detection by DHPLC. It is to be understood that a vast array of primers may be useful in the present invention, including those not specifically disclosed herein, without departing from the scope or preferred embodiments thereof.

The PCR primers of the instant invention are preferably designed or pre-selected to incorporate nucleotide sequences and/or reactive groups which will increase the melting temperature of an end section, or portion, of the amplicon. The present invention is based in part on Applicants surprising observation that the use of such primers, along with the nitrogen-containing compounds described herein, leads to improved mutation detection.

An example of a preferred method for increasing the midpoint temperature of a section of a PCR amplification product is the use of GC-clamp. (Myers et al., Nucleic Acids Res. 13:3111 (1985); Sheffield et al. (Proc. Natl. Acad. Sci. U.S.A 86:232-236 (1989)). CG-clamping is a technique in which additional G or C bases are included on the 5′ end of one or both of the PCR primers. The DNA polymerase enzyme will extend over these additional bases incorporating them into the amplicon thereby raising the midpoint temperature of the end(s) of the amplicon relative to that toward the middle of the amplicon. The size of the CG-clamp can be up to 100 bp and as little as 4 or 5 bp. The most preferred CG-clamp for mutation detection by DHPLC is 10 to 20 bp. In one embodiment, one primer in a set of primers for use in PCR incorporates a sequence comprising solely GC content on the 5′ end. The GC containing sequence can be up to 100 bp in length, preferably up to 40 bp, and more preferably between about 4 and 20 bp. The end terminal sequence can contain solely C, solely G, or solely CG, but preferably incorporates a clamp having both C and G.

Another method for increasing the midpoint temperature of a section of an amplicon includes incorporation of crosslinking agents. An example of a crosslinking agent is psoralen, which can be incorporated into one or more primers and used to crosslink adjacent strands, as disclosed in U.S. Pat. No. 5,652,096.

As described in the Examples herein (and as described by Narayanaswami et al. Genetic Testing 5:9-16 (2001)), the addition of a 20-base GC-clamp to a DNA fragment enabled mutations to be detected by denaturing high performance liquid chromatography (DHPLC) in the higher melting domain of the two-domain fragment DYS271. The mutations were undetectable in the absence of the GC-clamp.

In a general aspect of the invention, in preparing fragments for mutation detection by DHPLC, when PCR amplification is performed using primers designed to incorporate a GC-clamp into the amplification product, and when any of the nitrogen-containing compounds as described herein is present during the subsequent hybridization process in an effective concentration, mutations in DNA fragments can be detected which are otherwise undetectable.

Buffering agents and salts are used in the present invention to provide appropriate stable pH and ionic conditions for nucleic acid synthesis, e.g., for DNA polymerase activity, and for the hybridization process. A wide variety of buffers and salt solutions and modified buffers are known in the art that may be useful in the present invention, including agents not specifically disclosed herein. Preferred buffering agents include, but are not limited to, TRIS, TRICINE, BIS-TRICINE, HEPES, MOPS, TES, TAPS, PIPES, CAPS. Preferred salt solutions include, but are not limited to solutions of; potassium acetate, potassium sulfate, ammonium sulfate, ammonium chloride, ammonium acetate, magnesium chloride, magnesium acetate, magnesium sulfate, manganese chloride, manganese acetate, manganese sulfate, sodium chloride, sodium acetate, lithium chloride, and lithium acetate.

In another aspect, the present invention encompasses kits for use in detecting mutations in a double stranded DNA fragment. The kits may comprise one or more of the following: instructional material; a container that contains one or more of the nitrogen-containing compounds described herein; a container which contains one or more PCR primers wherein at least one of the primers includes a 5′ end-sequence of solely C, solely, G or solely GC nucleotide residues; a container which contains one or more PCR primers wherein at least one of the primers includes a crosslinking moiety; a container which contains a DNA polymerase; a container which contains a mutation standard; a container which contains wild type DNA corresponding to the DNA fragment; a container which contains buffer for carrying out a hybridization procedure. The kits can also contain one or more of a separation column (e.g. a reverse phase separation column or an ion exchange separation column) for use in separating DNA molecules; a liquid chromatography system; software for operating the chromatography system; software for analyzing data generated from the liquid chromatographic analysis of the DNA molecules; and software for analyzing and modeling the melting properties of DNA molecules (i.e. primer design software).

In one example of the practice of the invention, prior to the PCR amplification, a sample of double stranded DNA is mixed with corresponding wild type DNA. A section of the sample double stranded DNA is amplified simultaneously with the added wild type DNA. The PCR primers are designed such that all of the amplification products include a GC-clamp. The amplification product is subjected to hybridization as described herein.

In another example of the practice of the invention, a wild type double stranded DNA fragment corresponding to the sample of double stranded DNA, and including a CG-clamp of the same sequence as the amplified sample DNA, is added to the sample of double stranded DNA prior to the hybridization process described herein.

In still another example of the practice of the invention, a sample of double stranded DNA is obtained and amplified by PCR using a set of PCR primers in which at least one primer of the set includes a 5′ terminal sequence comprising solely GC content, whereby at least one CG-clamp is incorporated into the amplification product. If the sample is from a diploid organism which is heterozygous for the mutation, then the sample itself already contains both the wild type DNA and the DNA containing a single nucleotide polymorphism or other mutation. In this case, all of the amplification products will include the same GC clamp, and no exogenous (i.e. external) wild type DNA need be added prior to PCR or the hybridization process.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All patent applications, patents, and literature references cited in this specification are hereby incorporated by reference in their entirety. In case of conflict or inconsistency, the present description, including definitions, will control. Unless mentioned otherwise, the techniques employed or contemplated herein are standard methodologies well known to one of ordinary skill in the art. The materials, methods and examples are illustrative only and not limiting.

All numerical ranges in this specification are intended to be inclusive of their upper and lower limits.

Other features of the invention will become apparent in the course of the following descriptions of exemplary embodiments which are given for illustration of the invention and are not intended to be limiting thereof.

In the Examples provided herein, Applicants have demonstrated a quantitative study of a DNA fragment in which computer modeling predicted two distinct melting domains of approximately equal length. A mutation was introduced into the higher melting domain, and the fragment was subjected to hybridization with the original variant and analysis by DHPLC.

The heteroduplex yield was greatly decreased by the presence of mutations in the high melting domain. Without wishing to be bound by theory, Applicants believe that this is because this region anneals first during cooling, leading to selection of the more stable homoduplexes.

Only when a GC-clamp was added and the hybridization performed in the presence of a nitrogen-containing compound as described herein did the mutation become detectable by DHPLC. Without wishing to be bound by theory, Applicants believe that these nitrogen-containing organic compounds may act by suppressing the sequence-dependent melting behavior during the hybridization process.

Procedures described in the past tense in the Examples below have been carried out in the laboratory. Procedures described in the present tense have not yet been carried out in the laboratory, and are constructively reduced to practice with the filing of this application.

### EXAMPLE 1

### Thermocycler Program and Analysis Conditions

PCR was performed on an M&J PTC-200 thermocycler using a “touchdown” protocol to minimize nonspecific products (Don et al., 1991). In this approach, the annealing temperature is progressively lowered for a number of cycles to ensure that primers anneal most stringently in the early cycles. An initial denaturation step of 12 min at 95° C. to activate the polymerase was followed with 17 cycles of 20 sec at 94° C., 1 min annealing at 63-55° C. (the temperature decreasing by 0.5° C. per cycle), and 1 min at 72° C. After the touchdown stage, an additional 23 cycles were performed with 20 sec at 94° C., 1 min at 55° C., and 1 min at 72° C. in the main amplification stage. The program was completed with 5 min at 72° C. for quantitative extension and then storage at 4° C. (Kuklin et al., 1998).

### EXAMPLE 2

Preparation of 209-bp Variants 30C-44A-168A and 30C-44A-168G

PCRs were performed in a total volume of 100 μL containing 50 ng of plasmid with an insert containing variant 168A, 100 μM of each of the dNTPs, 1 μM of both sense and antisense primers, and 2.5 U/reaction of Amplitaq Gold™ DNA Polymerase (PE-Roche Molecular Systems, Branchburg, N.J.) in the buffer provided by the manufacturer. Sense primer of sequence 5′CAC TGG TCA GM TGA AG (SEQ ID NO: 2) and antisense primer of sequence 5′-MT GGA AAA TAC AGC TCC CC (SEQ ID NO: 3) were purchased from Operon Technologies.

### EXAMPLE 3

### Preparation and Cloning of Mutant 30T-44G-168A variant

One of the PCR products prepared above is denoted as 30C-44A-168A indicating the bases at the three variable locations. It was purified using DHPLC (WAVE®) DNA Fragment Analysis System from Transgenomic) to remove the sense and antisense primers and dNTPs associated with the reaction mixture. The purified fragment was then amplified with a 50-mer sense primer (5′-MGCACTGGTCAGMTGMGTGMTGGCATACAGGACMGTCCGGACCCA) (SEQ ID NO: 4) with two mutations compared with SEQ ID NO: 1 template, C→T at position 30 and a A→G at position 44. The antisense primer was the same as described in Example 2. After this step, the PCR product was cloned by a protocol described previously (Shaw-Bruha et al., Biotechniques 28:794-797 (2000)).

The new 30T-44G-168A variant contained two additional mutations with respect to the original 30C-44A-168A variant and three mutations with respect to the 30C-44A-168G variant.

### EXAMPLE 4

### Introduction of GC-clamps

PCRs were carried out as described in Example 3, except for the following modification. The sense primer was replaced with a 40-mer comprising 20 bases of solely GC content on the 5′ end of the regular sense primer 5′-CGCCCGCCGCCGCCCGCCGCAGGCACTGGTCAGAATGMG (SEQ ID NO: 5). The antisense primer was unchanged 5′-MTGGAAAATACAGCTCCCC (SEQ ID NO: 3). All primers were purchased from Operon Technologies.

### EXAMPLE 5

### Heteroduplex Formation

Hybridization was performed in which two variant PCR products (e.g., 30C-44A-168G with 30T-44G-168A) were mixed at equimolar ratios, heated to 95° C. for 4 min, and cooled down to 25° C. at a rate of 0.1 ° C. per 4 sec. The hybridization was performed after the completion of the PCR amplification and using the buffer described in Example 2.

Hybridizations in the presence of betaine (Sigma-Aldrich) were performed with equimolar concentration of each variant PCR product mixed with an equal volume of 6 M betaine (final concentration of 3 M betaine during the hybridization) and the mixture was subjected to the same hybridization conditions as described in the absence of betaine.

### EXAMPLE 6

Two variant PCR products are mixed at equimolar ratios, heated to 95° C. for4 min, and cooled down to 25° C. at a rate of 0.1° C. per4 sec. Hybridization in the presence of choline (Sigma-Aldrich) is performed with equimolar concentration of each variant PCR product mixed with an equal volume of 6 M choline (final concentration of 3 M choline in the hybridization mixture) and the mixture is subjected to the same hybridization conditions described in Example 5. The hybridization products are analyzed using the methods described in Example 10.

### EXAMPLE 7

Two variant PCR products are mixed at equimolar ratios, heated to 95° C. for 4 min, and cooled down to 25° C. at a rate of 0.1° C. per 4 sec. Hybridization in the presence of tetramethylammonium chloride (TMACl) (Sigma-Aldrich) is performed with equimolar concentration of each variant PCR product mixed with an equal volume of 6.6 M TMACl (final concentration of 3.3 M TMA in the hybridization mixture) and the mixture is subjected to the same hybridization conditions described in Example 5. The hybridization products are analyzed using the methods described in Example 10.

### EXAMPLE 9

Two variant PCR products are mixed at equimolar ratios, heated to 95° C. for 4 min, and cooled down to 25° C. at a rate of 0.1 ° C. per 4 sec. Separate hybridizations are performed, under the same hybridization conditions described in Example 5, in the presence of each of the following compounds (each at a final concentration of 2M): tetraethylammonium chloride (TEACl.), choline, dimethylglycine, sarcosine, stachydrine, trimethylamine N-oxide, and sulfobetaine. The hybridization products are analyzed using the methods described in Example 10.

### EXAMPLE 10

### Mutation Analysis

Mutations were detected in the hybridization products from the samples in Example 5 using DHPLC (WAVE® DNA Fragment Analysis System, Transgenomic, Inc., San Jose, Calif.). The hybridization mixture was injected (5 μL) automatically from the 96-well autosampler. The mobile phase buffers used for the separation were: Buffer A, 0.1 M triethylammonium acetate (TEM), pH 7.0 (Transgenomic Inc., San Jose, Calif.) in water; Buffer B, 0.1 M TEM and 25% acetonitrile in water pH 7.0. The elution of DNA fragments were monitored with a UV detector at 260 nm. Flow rate 0.9 ml min 21, gradient: 0 min, 65% A, 35% B; 1 min, 60% A, 40% B; 17.0 min, 28% A, 72% B; 17.1 min, 0% A, 100% B; 18.1 min, 0% A, 100% B; 18.2-20.1 min, equilibration at 65% A and 35% B.

### EXAMPLE 11

### Software Modeling

WAVEMAKER™ software (Transgenomic, Inc.) employing a Fixman-Friere algorithm (Fixman et al., Biopolymers 16:2963-2704 (1977)) parameterized specifically for DHPLC using the WAVE® System (Transgenomic) was used throughout.

The fragment used in this study was the 209-bp fragment from the human Y chromosome locus DYS271 (GenBank accession number S76940) with the sequence (Sites of sequence variants discussed herein are indicated in boldface.):

(SEQ ID NO:1)
AGGCACTGGTCAGAATGAAGTGAATGGCACACAGGACAAGTCCAGACCCA
GGAAGGTCCAGTAACATGGGAGAAGAACGGAAGGAGTTCTAAAATTCAGG
GCTCCCTTGGGCTCCCCTGTTTAAAAATGTAGGTTTTATTATTATATTTC
ATTGTTAACAAAAGTCCGTGAGATCTGTGGAGGATAAAGGGGGAGCTGTA
TTTTCCATT

The A→G transition in position 168 in SEQ ID NO: 1 was reported by Seielstad et al. (1994). The two sequence variants (168A and 168G) were prepared by PCR of cloned plasmids (kindly provided by Peter Underhill, Stanford University, Stanford, Calif., U.S.A) using the primers shown underlined. When the A and G variants were hybridized in the ratio x:y, they formed almost exactly the statistically expected ratio of species assuming nonstringent annealing (heteroduplex 1=xy, heteroduplex 2=yx, homoduplex 1=x²: homoduplex 2=y², or 1:1:1:1 in the case where x=y). The two heteroduplex species containing mismatched bases (A•C, G•T) and the two homoduplex species (A•T, G•C) appeared as four well-resolved peaks using DHPLC at 56-58° C. The four peaks were sufficiently well resolved that heteroduplexes were detectable when mutant to wild-type ratio is as low as 1 in 50 (Kuklin et al, 1998). The fragment 168G is available commercially (part no. 700210, Transgenomic) as a mutation standard to check instrument performance.

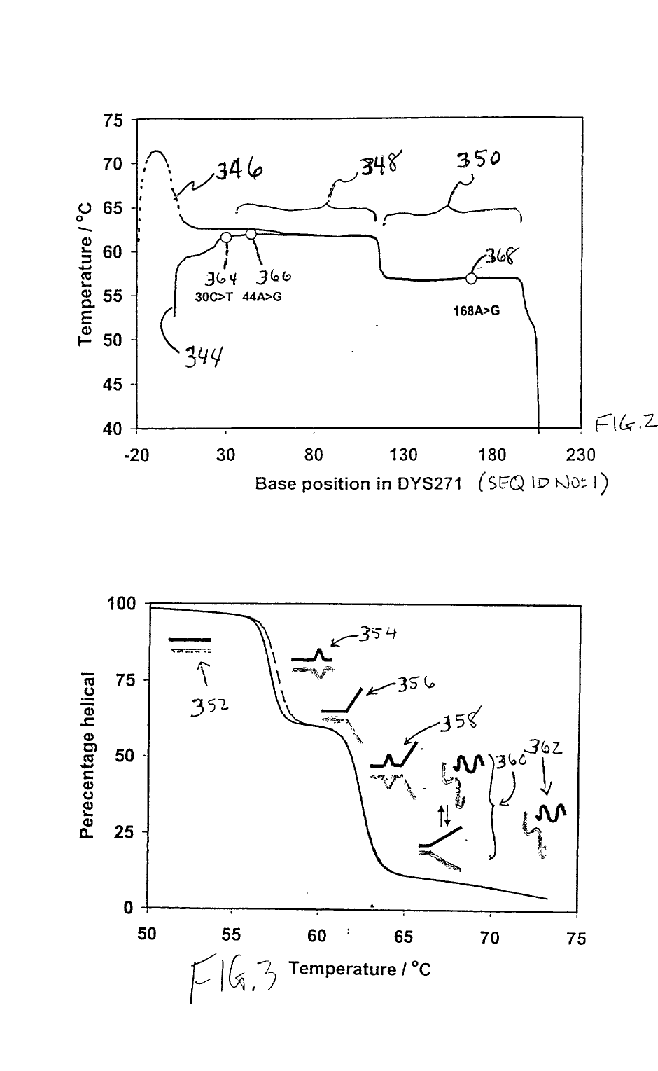

The melting properties of this fragment predicted using WAVEMAKER™ software are shown in FIG. 2 which shows a predicted melting map of homoduplex DYS271 (variant A) unclamped 344 (solid line) and with a 20-base GC-clamp attached 346 (dashed line). FIG. 2 shows the temperature at which each base has a 75% probability of being in the helical form. The locations and nature of the variant sequences in the high and low melting domains are indicated.

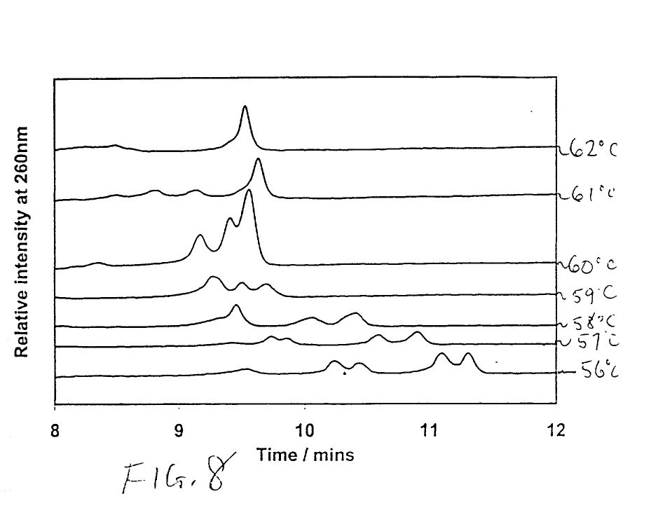

The bases from position 30-115 formed a high melting domain 348, which was predicted to be partially denatured (75% probability of bases being in the helical form), at 62° C. The bases from 120-195 formed a much lower melting domain as shown at 350, which was predicted to be partially denatured at 57° C. Mutation 168A→G is located in this lower melting domain. The average helicity of the GC-clamped 168G and 168A variants are shown in FIG. 3 which is annotated with schematic representation of the stages in process of denaturation (identified as 352, 354, 356, 358, 360, and 362 in the following discussion). The dashed line is the G variant whereas the solid line is the A variant (position 168, low melting domain). The predicted pattern is qualitatively similar to the experimental data shown in FIG. 7.

### EXAMPLE 11

### Effect of Betaine and GC-clamps

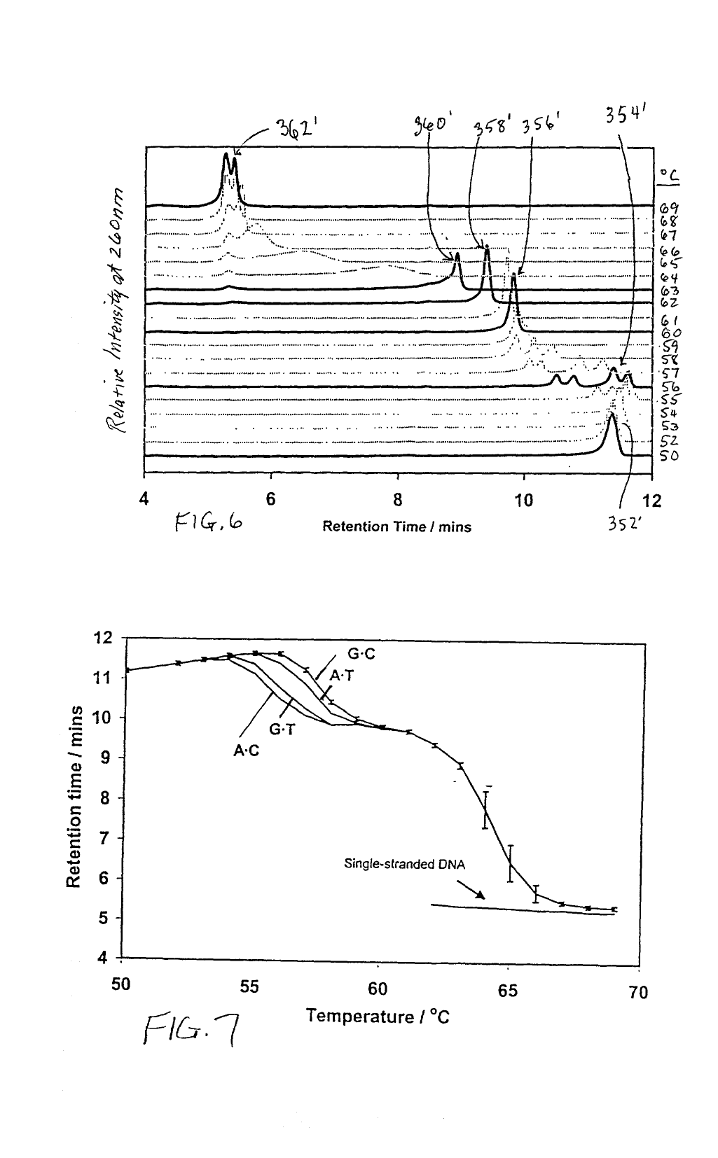

An illustration of a temperature titration is shown in FIG. 6 which shows chromatograms of GC-clamped, hybridized samples of the 168A and the 168G variants at 50° C. and at 52-69° C. in 1° C. increments. In FIG. 6, the chromatograms in bold, identified as 352′, 354′, 356′, 358′, 360′, and 362′, correspond to melting stages predicted in FIG. 3.

Chromatogram 352′ corresponds to fully doublestranded DNA. Chromatogram 354′ corresponds to strands partially denatured in the low melting domain. Chromatogram 356′ corresponds to fully denatured low melting domain. Chromatogram 358′ corresponds to partially denatured high melting domain. Chromatogram 360′ corresponds to a stage in which the GC clamp is the only remaining double-stranded region. Chromatogram 362′ corresponds to completely denatured single-stranded DNA.

In order to test whether mutations in the higher melting domain could be detected under conditions that are partially denaturing for this domain (60-63° C.), Applicants created and cloned (Example 3) a new insert from a PCR product obtained using the 168A plasmid with a 50-mer primer in which two mutations had been introduced leading to a 30C→T mutation shown at 364 and a 44A→G mutation shown at 366.

Hybridization of the original G variant of DYS271 (30C-44A-168G) with the site-directed mutagenesis product (30T-44G-168A) led to a mixture of two homoduplexes and two heteroduplexes. Each heteroduplex had a total of three mismatches, two in the higher melting domain and one in the lower melting domain shown at 368 (FIG. 2). The DHPLC analysis of this hybridization product was first compared with that of the original DYS271 mutation standard, which has just one mutation located in the low melting domain (168A→G). At 56° C., the temperature required to scan the low melting domain, the same four-peak pattern was observed with the hybridization product containing three mutations (30T -44G-168A +30C-44A-168G) as with the single mutation standard (30C-44A-168A +30C-44A-168G), indicating that the presence of mismatches in the higher melting domain has no effect on the resolution of the heteroduplex peaks in the low melting domain. However, the yield of the heteroduplexes was dramatically decreased by the presence of mismatches in the higher melting domain (this was observed both with and without a GC-clamp; the latter is shown in FIG. 5, chromatogram 370). At 60-63° C., the temperature range required to scan the high melting domain, a broad unresolved peak was obtained (FIG. 4, chromatogram 372) that was indistinguishable from the pure A variant, making it impossible to detect the presence of the two mutations in the higher melting domain using DHPLC.

At 60° C. the heteroduplexes resulting from the two mismatches in the higher melting domain (original DYS271 30C-44A-168G hybridized with the sequence variant 30T-44G-168A) were undetected within the broad peak (FIG. 4, chromatogram 372). Addition of a GC clamp but omission of betaine during hybridization led to a sharper peak but still no detectable heteroduplexes (FIG. 4, chromatogram 374). In contrast, use of a 20-base GC-clamp attached to the DYS271 fragment and a hybridization protocol that used 3 M betaine together led to straightforward detection of mutations within the high melting domain at 60° C. (FIG. 4, chromatogram 376).

FIG. 5 shows the quantitation of heteroduplex yields using the well-resolved heteroduplexes of the low melting domain mutation. When two mismatches were present in the high melting domain, the heteroduplexes resulting from the mismatch in the low melting domain (GC-clamped DYS271 30C-44A-168G hybridized with the sequence variant 30T-44G-168A) were well resolved at 56° C., but of low relative intensity (chromatogram 370). Modification of the hybridization protocol to include betaine (as shown by chromatogram 378) increased the yield of heteroduplex four-fold. When no mismatches were present in the higher melting domain (i.e. when the mutation was located in a low melting domain) the initial hybridization protocol produced essentially the statistically expected ratios of xy:yx:y^{2:}x² for heteroduplex1:heteroduplex2:homoduplex1:homoduplex2 (chromatogram 380) (Control).

### EXAMPLE 13

### Comparison of Software Modeling to Experimental Results

The extent to which the experimental results were predicted by the computer software model is now described. The mixture of the GC-clamped 168A and 168G variants, hybridized in betaine, gave the DHPLC chromatograms shown in FIG. 7 for temperatures 50-69° C. For simplicity, only the mutation in the low melting domain is shown in FIG. 7. A long linear gradient of 2% increase in Buffer B per minute was used to ensure that peaks remained within the linear section of the gradient, even at the highest temperature when the DNA is single stranded. The retention times of each peak were plotted in FIG. 7 against temperature in a “temperature titration” experiment. The overall two-domain melting behavior of the fragment predicted (FIGS. 2 and 3) was immediately apparent in the shape of the retention time versus temperature (FIG. 7). The steepest parts of both curves correspond to the midpoint of the melting of each domain at 57 and 63° C.

A number of interesting phenomena routinely observed in DHPLC were also apparent. The retention time for double-stranded DNA initially increased with temperature at about 0.13 min/° C. with the gradient used here (stage 352), until the onset of melting at about 54° C., at which point heteroduplexes just began to resolve. Thereafter, retention times steadily decreased with temperature. The four-peak pattern (two heteroduplexes and two homoduplexes) appeared between 56° C. and 57° C. as the low melting domain in which the sequence variants were located became partially denatured (stage 354). This pattern then collapsed and a plateau in retention time was reached, as the temperature was increased further (stage 356). The higher melting domain then started to denature partially (stage 358). However, before the high-melting domain became more denatured, peak broadening started to occur (FIG. 7) as the kinetically slow equilibrium with single-stranded DNA started to take over. At this point also, single-stranded DNA with a peak retention time of 5.4 min (FIG. 6) started to appear (stage 360). It is believed that this was formed during heating of the sample in passage to the column and the single strands were unable to re-anneal quickly enough to elute as double-stranded DNA. Finally the DNA was completely denatured and the forward and reverse strands eluted as separate peaks due to the sequence dependence given by the now-exposed hydrophobic bases (stage 362).

### EXAMPLE 14

### Effect of Betaine on Heteroduplex Formation

The pattern of peaks in FIG. 8 shows the equivalent experiment as described in EXAMPLE 12 with the GC-clamped DYS271 containing mismatches located in both the high and low melting domains (30C-44A-168A variant hybridized in 3 M betaine with the 30T-44G-168G variant). The mismatch in the lower melting domain is detected at 56-58° C., whereas the two mismatches in the higher melting domain are detected at 60° C. and 61° C. Heteroduplexes corresponding to the two mismatches located in the higher melting domain are very apparent from 60° C. to 61° C., corresponding to stage 358.

Without wishing to be bound by theory, Applicants believe that there were two factors operating to obscure the detection of heteroduplexes in the high melting domain of the DYS271 fragment:

1. The annealing of two sequences, which differ in a high melting region, appeared to occur with much greater stringency, leading to much reduced heteroduplex yield, than when the sequences differ in a low melting region.

2. The temperature at which the higher melting domain was partially denatured produced peaks which were too broad to resolve heteroduplexes.

The first factor can be attributed to the assumption that initiation of annealing occurs at the highest melting region. If the mutation happens to be located in this region, stringent annealing may occur, leading to selective formation of the more stable homoduplexes at the expense of heteroduplex formation. Hybridization in betaine or other nitrogen-compounds as described herein may act to suppress this stringent annealing and increase heteroduplex yield. Failure to form heteroduplexes in such cases may well be a cause for missed mutations.

The second factor can attributed to a kinetically slow equilibrium (on the chromatography time scale) with single-stranded DNA. As the helical content decreased at higher temperatures, the double-stranded DNA started to dissociate into single stranded DNA. Continuous, effectively irreversible dissociation to single-stranded DNA would take place during passage through the column, leading to peak broadening. Adding a GC-clamp to one or both ends of the fragment likely improved this by stabilizing double-stranded DNA at higher temperatures.

The GC-clamp consists of a sequence rich in G and C nucleotide of up to 40 bases in length and was introduced into a PCR product via the 5′ end of one primer. The GC-clamp stabilized the clamped end so that the remainder of the fragment could denature progressively to a Y-shaped structure. Hence, addition of a GC-clamp to one end of the DNA stabilized the DNA and raised the temperature at which the equilibrium shifted to single-stranded DNA.

Introducing a GC-clamp into the DYS271 fragment had the following effects. In either the GC-clamped or unclamped amplicons, the variant in the low melting domain 168A→G gave a distinct four-peak pattern at 56° C. However, the two mutations introduced into the high melting domain (30C→T and 44A→G) were not detectable in the unclamped form at any temperature because of the onset of peak broadening just prior to the temperature required to partially denature the domain. The GC-clamp maintained a sharp peak up to 63° C. compared with 59° C. in the absence of the clamp. This opened up a window in which the peaks remain sharp and the heteroduplexes could be readily detected (FIG. 4). Even the GC-clamped fragment underwent dramatic peak broadening after 63° C. (FIG. 7). Comparison of FIG. 3 and FIG. 7 suggests that this occurred when the fragment was 40% helical or when 83 of the 209 bases remained helical. Predicting the temperature at which peak broadening occurs allows amplicons to be designed such that the domain of interest can be partially denatured at a temperature below that at which peak broadening occurs. Although the desired melting behavior may also be achieved by repositioning primers to intrinsically high-melting regions upstream and downstream of the region of interest, this may not be appropriate in cases where the additional DNA sequence may contain mutations of lesser interest such as intronic DNA. Use of GC-clamp of a length of 15-25 bases provided a simple method of altering the melting behavior without repositioning the primers with respect to the template. A small effect of GC-clamps on the yield of PCR product was noticed and has been previously reported (McDowell et al., Nucleic Acids Res. 26:3340-3347 (1998)). According to the authors, a high %GC may result in either an overall stiffening effect on the helix or an increased chance of localized higher Tm regions with associated stiffness, which may slow polymerase extension. The additional cost per sample for a 20-base clamp is minimal.

While the foregoing has presented specific embodiments of the present invention, it is to be understood that these embodiments have been presented by way of example only. It is expected that others will perceive and practice variations which, though differing from the foregoing, do not depart from the spirit and scope of the invention as described and claimed herein.

All patent applications, patents, and literature references cited in this specification are hereby incorporated by reference in their entirety. In case of conflict or inconsistency, the present description, including definitions, will control.

The invention claimed is:

1. A method for preparing a double stranded DNA fragment for mutation detection by denaturing high performance liquid chromatography, the double stranded DNA fragment corresponding to a wild type double stranded DNA fragment having a known nucleotide sequence, the method comprising:
(a) amplifying a section of said double stranded DNA fragment for mutation detection by PCR, using a set of primers which flank said section, wherein at least one primer of said set incorporates a sequence comprising solely GC content on the 5′ end;
(b) hybridizing the amplification product of step (a) with wild type double stranded DNA corresponding to said section, whereby a mixture comprising one or more heteroduplexes is formed if said section includes a mutation; and
(c) including during said hybridizing an amount of a composition of the formula: 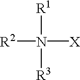
wherein:
R¹, R², and R³, may be the same or different and are independently selected from the group consisting of hydrogen, methyl, ethyl, hydroxyethyl, and propyl, with the proviso that no more than two of R¹, R², and R³ are hydrogen; and X is a moiety selected from the group consisting of:
radicals of the formulas
═O;
→O
—CH₃;
—CH₂CH₃; and 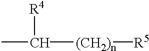
wherein:
R⁴ is selected from the group consisting of methyl and hydrogen and, when combined with R¹, forms a pyrrolidine ring;
R⁵ is selected from the group consisting of —CO₂H, —CH₂OH, and —SO₃H; and
n is an integer of from 0 to 2;
with the proviso that, when R¹ and R⁴ form a pyrrolidine ring, no more than one of R² and R³is hydrogen;
wherein the composition is included in an amount effective to increase the amount of heteroduplex DNA double stranded DNA fragment for mutation detection.

2. The method of claim 1 wherein R¹, R² and R³ are the same or different and selected from the group consisting of methyl, ethyl and hydrogen with the proviso that no more than two of R¹, R² and R³ are hydrogen and, when R¹ and R⁴ form a pyrrolidine ring, no more than one of R² and R³ is hydrogen.

3. The method of claim 2 wherein X is —CH₂CO₂H.

4. The method of claim 2 wherein X is —CH₂CO₂H and wherein R¹, R², and R³ are methyl.

5. The method of claim 3 wherein R¹, R² and R³ are methyl.

6. The method of claim 3 wherein R¹ and R² are methyl and R³is hydrogen.

7. The method of claim 3 wherein R¹ is methyl and R² and R³ are hydrogen.

8. The method of claim 2 wherein X is ═O.

9. The method of claim 8 wherein R¹, R² and R³ are methyl.

10. The method of claim 2 wherein R¹ and R⁴ form a pyrrolidine ring, R² and R³ methyl, n is 0, and R⁵is —CO₂H.

11. The method of claim 2 wherein R¹, R² and R³ are methyl and X is —CH₂SO₃.

12. The method of claim 1 wherein the composition comprises trimethylglycine.

13. The method of claim 1 wherein said composition is present at a concentration in the range of 1M to 8M.

14. The method of claim 13 wherein said liquid chromatography is carried out under conditions effective to at least partially denature said heteroduplexes.

15. The method of claim 1 wherein said double stranded DNA fragment for mutation detection comprises unpurified DNA.

16. The method of claim 15 wherein said unpurified DNA is a crude cell lysate.

17. The method of claim 1 wherein at least one primer of said set incorporates up to 40 bases of solely GC content on the 5′ end.

18. The product of the method described by claim 1.

19. The method of claim 1 wherein step (b) includes:
(i) heating the mixture of step (b) to a temperature at which the strands are completely denatured;
(ii) cooling the product of step (i) until the strands are completely annealed, whereby a mixture comprising one or more heteroduplexes is formed if said section includes a mutation.

20. A method for preparing a double stranded DNA fragment for mutation detection by denaturing high performance liquid chromatography, the double stranded DNA fragment corresponding to a wild type double stranded DNA fragment having a known nucleotide sequence, the method comprising:
(a) amplifying a section of said double stranded DNA fragment for mutation detection by PCR, using a set of primers which flank said section, wherein at least one primer of said set incorporates a sequence comprising solely GC content on the 5′ end;
(b) hybridizing the amplification product of step (a) with wild type double stranded DNA corresponding to said section, whereby a mixture comprising one or more heteroduplexes is formed if said section includes a mutation; and
(c) including during said hybridizing a compound of the formula: 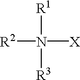
wherein:
R¹, R², and R³, may be the same or different and are independently selected from the group consisting of hydrogen, methyl, ethyl, hydroxyethyl, and propyl, with the proviso that no more than two of R¹, R², and R³ are hydrogen; and X is a moiety selected from the group consisting of:
radicals of the formulas
═O;
→O
—CH₃;
—CH₂CH₃; and 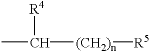
wherein:
R⁴ is selected from the group consisting of methyl and hydrogen and, when combined with R¹, forms a pyrrolidine ring;
R⁵ is selected from the group consisting of —CO₂H, —CH₂OH, and —SO₃H; and
n is an integer of from 0 to 2;
with the proviso that, when R¹ and R⁴ form a pyrrolidine ring, no more than one of R² and R³ is hydrogen; and wherein said compound is present at a concentration in the range of 1M to 8M during the hybridization of step (b).

21. A method for mutation detection of a double stranded DNA fragment by denaturing high performance liquid chromatography, the double stranded DNA fragment corresponding to a wild type double stranded DNA fragment having a known nucleotide sequence, the method comprising the steps of:
(a) amplifying a section of said double stranded DNA fragment by PCR using a set of primers which flank the ends of said section, wherein at least one primer of said set incorporates a sequence comprising solely GC content on the 5′ end;
(b) hybridizing the amplification product of step (a) with wild type double stranded DNA corresponding to said section, whereby a mixture comprising one or more heteroduplexes is formed if said section includes a mutation; and
(c) analyzing the product of step (b) by Denaturing High Performance Liquid Chromatography; and
(d) including during said hybridizing an amount of a composition of the formula: 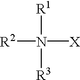
wherein:
R¹, R², and R³, may be the same or different and are independently selected from the group consisting of hydrogen, methyl, ethyl, hydroxyethyl, and propyl, with the proviso that no more than two of R¹, R², and R³ are hydrogen; and
X is a moiety selected from the group consisting of:
radicals of the formulas
═O;
→O
—CH₃;
—CH₂CH₃; and 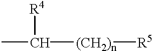
wherein:
R⁴ is selected from the group consisting of methyl and hydrogen and, when combined with R¹, forms a pyrrolidine ring;
R⁵ is selected from the group consisting of —CO₂H, —CH₂OH, and —SO₃H; and
n is an integer of from 0 to 2;
with the proviso that, when R¹ and R⁴ form a pyrrolidine ring, no more than one of R² and R³ is hydrogen;
wherein the composition is included in an amount effective to increase the amount of heteroduplex DNA.

22. The method of claim 21 wherein R¹, R² and R³are the same or different and selected from the group consisting of methyl, ethyl and hydrogen with the proviso that no more than two of R¹, R² and R³ are hydrogen and, when R¹ and R⁴ form a pyrrolidine ring, no more than one of R² and R³ is hydrogen.

23. The method of claim 22 wherein X is —CH—₂CO₂H.

24. The method of claim 22 wherein X is —CH—₂CO₂H and wherein R¹, R², and R³ are methyl.

25. The method of claim 23 wherein R¹, R² and R³ are methyl.

26. The method of claim 23 wherein R¹ and R² are methyl and R³ is hydrogen.

27. The method of claim 23 wherein R¹ is methyl and R² and R³ are hydrogen.

28. The method of claim 22 wherein X is ═O.

29. The method of claim 28 wherein R¹, R² and R³ are methyl.

30. The method of claim 21 wherein R¹ and R⁴ form a pyrrolidine ring, R² and R³ methyl, n is 0, and R⁵ is —CO₂H.

31. The method of claim 22 wherein R¹, R² and R³ are methyl and X is —CH₂SO₃.

32. The method of claim 21 wherein the composition comprises trimethylglycine.

33. The method of claim 21 wherein wherein said composition is present at a concentration in the range of 1M to 8M.

34. The method of claim 21 wherein said double stranded DNA fragment for mutation detection comprises unpurified DNA.

35. The method of claim 34 wherein said unpurified DNA comprises a crude cell lysate.

36. The method of claim 21 wherein at least one primer of said set incorporates up to 40 bases of solely GC content on the 5′ end.

37. The method of claim 21 wherein step (b) includes:
(i) heating the mixture of step (b) to a temperature at which the strands are completely denatured;
(ii) cooling the product of step (i) until the strands are completely annealed, whereby a mixture comprising one or more heteroduplexes is formed if said section includes a mutation.

38. A kit for preparing a double stranded DNA for mutation detection by liquid chromatography, said kit comprising:
(a) a container which contains one or more PCR primers, wherein at least one primer of said set incorporates a sequence comprising solely GC content on the 5′ end;
and (b) a composition of the formula: 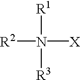
wherein:
R¹, R², and R³, may be the same or different and are independently selected from the group consisting of hydrogen, methyl, ethyl, hydroxyethyl, and propyl, with the proviso that no more than two of R¹, R², and R³ are hydrogen; and X is a moiety selected from the group consisting of: radicals of the formulas
═O;
→O
—CH₃;
—CH₂CH₃; and 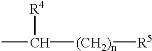
wherein:
R⁴ is selected from the group consisting of methyl and hydrogen and, when combined with R¹, forms a pyrrolidine ring;
R⁵ is selected from the group consisting of —CO₂H, —CH₂OH, and —SO₃H; and
n is an integer of from 0 to 2;
with the proviso that, when R¹ and R⁴ form a pyrrolidine ring, no more than one of R² and R³ is hydrogen.

39. The kit of claim 38 wherein R′, R² and R³ are the same or different and selected from the group consisting of methyl, ethyl and hydrogen with the proviso that no more than two of R¹, R² and R³ are hydrogen and, when R¹ and R⁴ form a pyrrolidine ring, no more than one of R² and R³ is hydrogen.

40. The kit of claim 38 wherein X is —CH—₂CO₂H.

41. The kit of claim 38 wherein X is —CH—₂CO₂H and wherein R¹, R², and R³ are methyl.

42. The kit of claim 41 wherein R¹, R² and R³ are methyl.

43. The kit of claim 41 wherein R¹ and R² are methyl and R³ is hydrogen.

44. The kit of claim 41 wherein R¹ is methyl and R² and R³ are hydrogen.

45. The kit of claim 38 wherein X is ═O.

46. The kit of claim 45 wherein R¹, R² and R³ are methyl.

47. The kit of claim 38 wherein R¹ and R⁴ form a pyrrolidine ring, R² and R³ methyl, n is 0, and R⁵ is —CO₂H.

48. The kit of claim 38 wherein R¹, R² and R³ are methyl and X is —CH₂SO₃ and wherein the concentration of said composition in said kit is such that the final concentration of said composition in a hybridization procedure is in the range of 1 to 8M.

49. The kit of claim 38 wherein the composition comprises trimethylglycine.

50. The kit of claim 38 further comprising wild type double stranded DNA corresponding to said double stranded DNA for mutation detection.

51. The kit of claim 38 wherein the kit comprises a DNA polymerase.

52. The kit of claim 51 wherein the kit comprises a proofreading DNA polymerase.

53. The kit of claim 52 wherein the kit comprises Pho polymerase.

54. The kit of claim 52 wherein the kit comprises Taq polymerase.

55. A kit for hybridizing a target nucleotide sequence with wild type DNA corresponding to said target sequence, said kit comprising in separate containers:
(a) wild type DNA corresponding to said target sequence, and
(b) trimethylglycine.

56. The kit of claim 55 wherein the nucleotide sequence being hybridized is indicative of a disease state.

57. The kit of claim 55 including in a separate container PCR primers for amplifying said target sequence, wherein at least one primer of said set incorporates a sequence comprising solely GC content on the 5′ end.

58. A kit for analyzing a double stranded DNA for mutation detection by liquid chromatography, said kit comprising:
(a) wild type DNA corresponding to said double stranded DNA,
(b) a reverse phase separation medium,
(c) a DNA polymerase; and
(d) trimethylglycine.

59. The kit of claim 58 wherein the kit further comprises wild type DNA corresponding to said double stranded DNA.

60. The kit of claim 59 wherein the kit further comprises at least one mutation standard.

61. In an improved method for hybridizing a target double stranded DNA with corresponding wild type double stranded DNA, the improvement comprising: adding an effective amount of trimethylglycine to the hybridization mixture wherein more heteroduplex molecules are produced than would be produced in the absence of said trimethylglycine.

62. The procedure of claim 61 wherein the target double stranded DNA being amplified is indicative of a disease state.

63. A method for preparing a double stranded DNA fragment for mutation detection by denaturing high performance liquid chromatography, the double stranded DNA fragment, the method comprising:
(a) amplifying a section of said double stranded DNA fragment for mutation detection by PCR, using a set of primers which flank said section, wherein at least one primer of said set incorporates up to 40 bases of solely GC content on the 5′ end;
(b) in the presence of betaine, hybridizing the amplification product of step
(a) with wild type DNA corresponding to said section.

64. The method of claim 63 wherein step (b) includes:
(i) heating the mixture of step (b) to a temperature at which the strands are completely denatured;
(ii) cooling the product of step (i) until the strands are completely annealed, whereby a mixture comprising one or more heteroduplexes is formed if said section includes a mutation.
